(19) United States
(12) Patent Application Publication
(10) Pub. No.: US 2019/0115093 A1
(43) Pub. Date: Apr. 18, 2019

(54) ANNOTATING DATA POINTS ASSOCIATED WITH CLINICAL DECISION SUPPORT APPLICATION

(71) Applicant: KONINKLIJKE PHILIPS N.V., EINDHOVEN (NL)

(72) Inventors: Reza FIROOZABADI, THOUSAND OAKS, CA (US); Eric HELFENBEIN, SUNNYVALE, CA (US); Saeed BABAEIZADEH, ARLINGTON, MA (US)

(73) Assignee: KONINKLIJKE PHILIPS N.V., EINDHOVEN (NL)

(21) Appl. No.: 16/092,012

(22) PCT Filed: Apr. 5, 2017
(86) PCT No.: PCT/EP2017/058048

§ 371 Date: Oct. 8, 2018

Related U.S. Application Data
(60) Provisional application No. 62/323,181, filed on Apr. 15, 2016.

(51) Int. Cl. G16H 10/60; G06F 16/28; G16H 50/30

(52) U.S. Cl. CPC G16H 10/60; G16H 50/30; G06F 16/285

(57) ABSTRACT

In various embodiments, a plurality of inputs for a CDS algorithm that is executable by one or more processors may be identified (602). A plurality of health parameters associated with a patient-of-interest may be obtained (604) for use as at least some of the plurality of inputs for the CDS algorithm. One or more of the plurality of inputs for which a corresponding health parameter associated with the patient is out-of-date or unavailable may be identified as suboptimal (606). The CDS algorithm may then be executed (616) using the plurality of health parameters as input. Output indicative of a result of the CDS algorithm may be rendered (618). In various embodiments, the output may present at least one data point associated with the suboptimal one or more inputs more conspicuously than one or more data points associated with other inputs, e.g., using visual or audible annotations.

### TECHNICAL FIELD

Various embodiments described herein are directed generally to health care. More particularly, but not exclusively, various methods and apparatus disclosed herein relate to visually and/or audibly annotating one or more input or output data points associated with execution of a clinical decision support (“CDS”) application.

### BACKGROUND

CDS software applications may be implemented in numerous health care settings (e.g., care information systems) in order to aid medical personnel in making a variety of clinical decisions. One example CDS algorithm that is used to calculate acute kidney injury (“AKI”) takes into account health parameters such as patient demographics, serum creatinine, and urine output. At the time of execution, CDS applications may obtain, as inputs, various health parameters associated with a patient from different sources, and may calculate various CDS metrics based on these inputs. However, some health parameters may be unavailable, either because they have never been collected or because they are out-of-date to a degree that they have expired (e.g., a blood pressure reading from two years ago may not have probative value). Other health parameters may be available to be used as input, but may be out-of-date to a degree that they are “stale” but still useable. In order to execute a CDS application in spite of one or more health parameters being unavailable or expired, the unavailable or expired health parameters may be replaced with various values, such as a population mean value for a given health parameter. However, executing a CDS algorithm with such replacement values impacts the reliability of the algorithm output. Additionally, when a CDS application is executed using one or more stale health parameters as input, the output of the CDS application is also potentially less reliable.

### SUMMARY

The present disclosure is directed to various methods and apparatus for visually and/or audibly annotating one or more input or output data points associated with execution of a CDS application. For example, suppose a CDS software application is executed on a computing system using one or more stale or replacement health parameters as input. In various embodiments, the CDS application may render output such that one or more data points directly or indirectly affected by the unavailable and/or out-of-date health parameters are visually and/or audibly annotated. That way, a user is made aware of the fact that certain data points, such as input values or output values, should be viewed with heightened scrutiny.

Generally, in one aspect, a computer-implemented method may include: identifying, by one or more processors, a plurality of inputs for a CDS application that is executable by the one or more processors; obtaining, by the one or more processors, a plurality of health parameters associated with a patient to be used as at least some of the plurality of inputs for the CDS application; identifying as suboptimal, by the one or more processors, one or more of the plurality of inputs for which a corresponding health parameter associated with the patient is out-of-date or unavailable; executing, by the one or more processors, the CDS application using the plurality of health parameters as input; and rendering, by the one or more processors, output indicative of a result of the CDS application, wherein the output presents at least one data point associated with the suboptimal one or more inputs more conspicuously than one or more data points associated with other inputs.

In various embodiments, the identifying as suboptimal may include classifying a given health parameter of the patient as out-of-date or unavailable based on a comparison of a timestamp associated with the given health parameter with one or more time intervals. In various versions, the one or more time intervals may include a stale time interval, and the given health parameter is classified as stale when the timestamp associated with the given health parameter falls within the stale time interval. In various versions, a health parameter classified as stale may be used as input for the CDS application. One or more data points associated with the stale health parameter may be presented in a manner that reveals the stale classification.

In various versions, the one or more time intervals may include a fresh time interval that follows the stale time interval. The given health parameter may be classified as fresh when the timestamp associated with the given health parameter falls within the fresh time interval. In various versions, a health parameter classified as fresh may be used as input for the CDS application. One or more data points associated with the fresh health parameter may be presented in a manner that differs from the stale health parameter.

In various embodiments, the one or more time intervals may include an expired time interval that precedes the stale time interval. The given health parameter may be classified as expired when the timestamp associated with the given health parameter falls within the expired time interval. In various versions, a substitute value may be used as input for the CDS application in place of a health parameter classified as expired. One or more data points associated with the expired health parameter may be presented in a manner that reveals the expired classification. In various versions, the substitute value may include a population mean or a range of values that encompasses the population mean.

In various embodiments, the identifying as suboptimal may include classifying a given health parameter of the patient as unavailable based on a determination that the given health parameter has not been measured. A substitute value or range of substitute values may be used as input for the CDS application in place of a health parameter classified as unavailable. One or more data points associated with the unavailable health parameter may be presented in a manner that reveals the unavailable classification.

In various embodiments, the output may visually annotate the at least one data point associated with the one or more suboptimal inputs. In various versions, the output visually annotates the at least one data point with highlighting, font selection, animation, or extra characters. In various versions, the at least one data point associated with the one or more suboptimal inputs includes a value computed by the CDS application based at least in part on the one or more suboptimal inputs. In various versions, the identifying as suboptimal may include classifying a given health parameter of the patient as expired or unavailable. In some such embodiments, a substitute range of values may be used as input for the CDS application in place of the given health parameter.

Other embodiments may include a non-transitory computer readable storage medium storing instructions executable by a processor to perform a method such as one or more of the methods described above. Yet another implementation may include a system including memory and one or more processors operable to execute instructions, stored in the memory, to implement a method such as one or more of the methods described above.

As used herein, a “clinical decision support” algorithm is a procedure or formula for providing health care personnel with assistance with clinical decision making. In particular, clinical decision support systems are defined by some as “active knowledge systems which use two or more items of patient data to generate case-specific advice.” A CDS application is a software application that may be executed by one or more processors of a computing system to take, as input, one or more health parameters of a patient, and to provide output that aids medical personnel in making one or more clinical decisions. CDS applications may employ a variety of knowledge- and non-knowledge-based techniques such as supervised or unsupervised machine learning models, neural networks, heuristic models, and so forth.

“Items of patient data,” or as they are referred to herein, “health parameters,” may include but are not limited to a patient's age, weight, gender, blood pressure, pulse rate, lab results, temperature, glucose levels, blood oxygen saturation level (“SpO2”), tidal volume (“Vt”), fraction of inspired oxygen (“FiO2”), positive end-expiratory pressure (“PEEP”), partial pressure of oxygen in blood (“PaO2”), peak inspiratory pressure (“PIP”), endotracheal tube (“ETT”) placement, partial pressure of carbon dioxide (“EtCO2”), and so forth.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1 schematically illustrates an example environment in which disclosed techniques may be employed in accordance with various embodiments.

FIGS. 2-5 depict example user interfaces configured with selected aspects of the present disclosure.

FIG. 6 depicts an example method, in accordance with various embodiments.

FIG. 7 depicts an example timeline with various predetermined time intervals defined, in accordance with various embodiments.

FIG. 8 depicts components of an example computing system.

### DETAILED DESCRIPTION

CDS software applications may aid medical personnel in making a variety of clinical decisions. At the time of execution, CDS applications may obtain, as inputs, various health parameters associated with a patient from different sources, and may calculate various CDS metrics or “results” based on these inputs. However, some health parameters may be unavailable, either because they have never been collected or because they are expired. Other health parameters may be available to be used as input, but may not be ideal because they are stale. In order to execute a CDS application in spite of one or more health parameters being unavailable or expired, the unavailable or expired health parameters may be replaced with various values, such as a population mean value for a given health parameter. However, executing a CDS algorithm with such replacement values impacts the reliability of the algorithm output. Additionally, when a CDS application is executed using one or more stale health parameters as input, the output of the CDS application is also potentially less reliable. In view of the foregoing, various embodiments and implementations of the present disclosure are directed to visually and/or audibly annotating one or more input or output data points associated with execution of a CDS application in order to place users on notice that the annotated data points may warrant heightened scrutiny, e.g., due to their potentially being less reliable than perhaps expected.

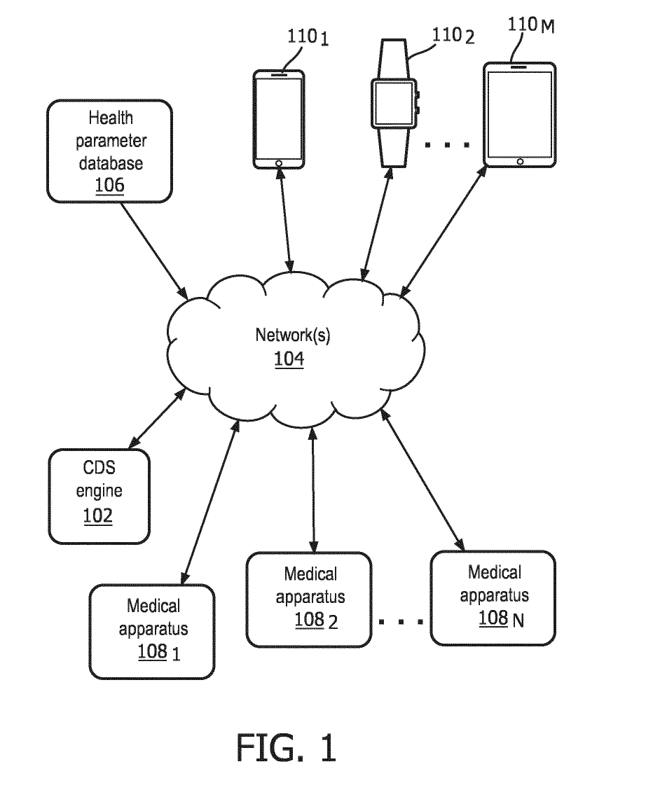

Referring to FIG. 1, in one embodiment, an environment in which disclosed techniques may be employed may include a CDS engine 102. CDS engine 102 may include one or more computing systems (with standard computing components) communicatively coupled with other components depicted in FIG. 1 via one or more networks 104. One or more networks 104 may include one or more wired or wireless local area networks (“LANs”) and/or one or more wired or wireless wide area networks (“WANs”) such as the Internet.

One component that may be in communication with CDS engine 102 is a health parameter database 106. In various embodiments, health parameter database 106 may store records of observed and/or observable health parameters associated with a plurality of patients. For example, health parameter database 106 may include a plurality of patient records that include, among other things, data indicative of one or more health parameters of the patients. Example health indicators are described above.

The term “database” as used herein refers to a collection of data and information organized in such a way as to allow the data and information to be stored, retrieved, updated, and manipulated and to allow them to be presented into one or more formats such as in table form or to be grouped into text, numbers, images, and audio data. The term “database” as used herein may also refer to a portion of a larger database, which in this case forms a type of database within a database. “Database” as used herein also refers to conventional databases that may reside locally or that may be accessed from a remote location, e.g., remote network servers. The database typically resides in computer memory that includes various types of volatile and non-volatile computer memory. Memory that stores the database may include high-speed random access memory or non-volatile memory such as magnetic disk storage devices, optical storage devices, and flash memory. Memory that stores the database resides may also store one or more software for processing and organizing data received by and stored into the database.

CDS engine 102 also may be in communication with one or more medical apparatus 108_{1-N}. Medical apparatus 108_{1-N} may be configured to obtain (e.g., measure) various health parameters from patients, and therefore may come in various forms, including but not limited to electrocardiogram (“EKG”) machines, X-ray machines, blood sugar monitoring machines, blood pressure readers, temperature sensors, and so forth. CDS engine 102 also may be in communication with one or more computing devices 110_{1-M}, that are configured to present CDS results and metrics calculated using various CDS algorithms. Computing devices 110 may come in various form factors including but not limited to smart phones (e.g., 110₁), wearable devices (e.g., smart watch 110₂), tablet computers (e.g., 110_{M}), laptop computers, desktop computers, set top boxes, and so forth.

In various embodiments, CDS engine 102 may include one or more processors (not depicted in FIG. 1; see FIG. 8) that are configured to execute instructions stored in memory (not depicted in FIG. 1, see FIG. 8) that cause the one or more processors to identify a plurality of inputs for a CDS software application that is executable by CDS engine 102. For example, in one CDS algorithm repeatedly described herein for illustrative purposes, the expected inputs include SpO2, Vt (Spontaneous), FiO2, PEEP, and PaO2. Of course, these health parameters and different health parameters may be used in different combinations to compute different CDS metrics/results. CDS engine 102 may be further configured to obtain, e.g., from health parameter database 106 and/or one or more medical apparatus 108, a plurality of health parameters associated with a patient (not depicted) to be used as at least some of the plurality of inputs for the CDS application.

CDS engine 102 may then analyze the obtained health parameters to identify as suboptimal (e.g., by flagging in memory) one or more of the plurality of inputs for which a corresponding health parameter associated with the patient is out-of-date or unavailable. For example, in some embodiments, CDS engine 102 may classify a given health parameter associated with a patient as out-of-date or unavailable based on a comparison of a timestamp associated with the given health parameter with one or more time intervals. If, for instance, the health parameter was measured more than six hours in the past, the health parameter may be classified as “expired,” and may not be used as input for a CDS algorithm. If the health parameter was measured between two and six hours in the past, the health parameter may be classified as “stale.” That may mean the health parameter is still usable, but that the CDS results should be subjected to heighted scrutiny. If the health parameter was measured less than two hours in the past, it may still be considered out-of-date because it is not measured contemporaneously with the CDS calculation, but still may be considered “fresh” in that it may be safely used without much, if any, heightened scrutiny. If a health parameter was measured concurrently or at least contemporaneously with a current CDS calculation, that health parameter may be classified “current.”

Once the one or more inputs are identified as suboptimal, CDS engine 102 may execute the CDS application using at least some of the plurality of health parameters (e.g., those that are not expired or unavailable) as input. In various embodiments, health parameter inputs classified as unavailable or expired may be replaced with various values. In some embodiments, unavailable/expired health parameters may be replaced with population averages for that health parameter. For example, if a patient's blood pressure is unavailable, or has not been taken for so long that the last reading is not considered probative of the patient's current health, then the population average of 120/80 may be used instead as input for the CDS application. In some embodiments, the germane population may be selected based at least in part on other health parameters associated with the patient that are available/fresh/unaffected by time. For example, if the patient's blood pressure is unavailable but the patient's weight was recently measured to be at a level considered to be obese and the patient is male, then a mean blood pressure among a population consisting of obese males may be substituted for the patient's actual blood pressure. In some embodiments, rather than using a population mean as a substitute for an unavailable or expired health parameter, a range of values may be substituted. For example, a range from one standard deviation below a population mean to one standard deviation above the population may be used as a substitute for an unavailable or expired health parameter.

Once the CDS application is executed with the obtained health parameters and substituted values, CDS engine 102 may render, or cause to be rendered on one or more computing devices 110, output indicative of one or more results of the CDS algorithm. In various embodiments, the output may present at least one data point associated with the one or more suboptimal inputs more conspicuously than one or more data points associated with other inputs. In this manner, it may be revealed to users such as medical personnel that view the CDS results that certain input and/or output data points are stale, unavailable, expired, substituted, or calculated based on stale and/or substituted data points. With this knowledge, the medical personnel may view the various metrics with an appropriate level of scrutiny.

While CDS engine 102 is shown as a standalone component in FIG. 1, this is not meant to be limiting. In various embodiments, one or more selected aspects of CDS engine 102 may be implemented elsewhere, e.g., on one or more computing devices 110. For example, CDS engine 102 may gather the health parameters to be used as input for a given CDS application, and may provide those health parameters to a computing device 110. Once it receives the health parameters, computing device 110 may execute a local CDS client to cause one or more disclosed techniques to be performed. Additionally or alternatively, in some embodiments, CDS engine 102 may be entirely implemented on a computing device 110 operated by a medical personnel, such that the computing device 110 obtains valid health parameters, determines substitute values for expired/unavailable health parameters, and executes the CDS application. In some embodiments, CDS engine 102 may provide a web interface, and users may interact with the CDS engine 102 by operating web browsers to load interactive web pages that present annotated data points as described herein. For example, CDS engine 102 may generate a user interface document such as a hypertext markup language (“HTML”) or extensible markup language (“XML”) document and transmit it to a computing device 110 to be presented to a user operating a web browser. In some embodiments, standard HTML/XML tags may be used to annotate selected data points as described herein.

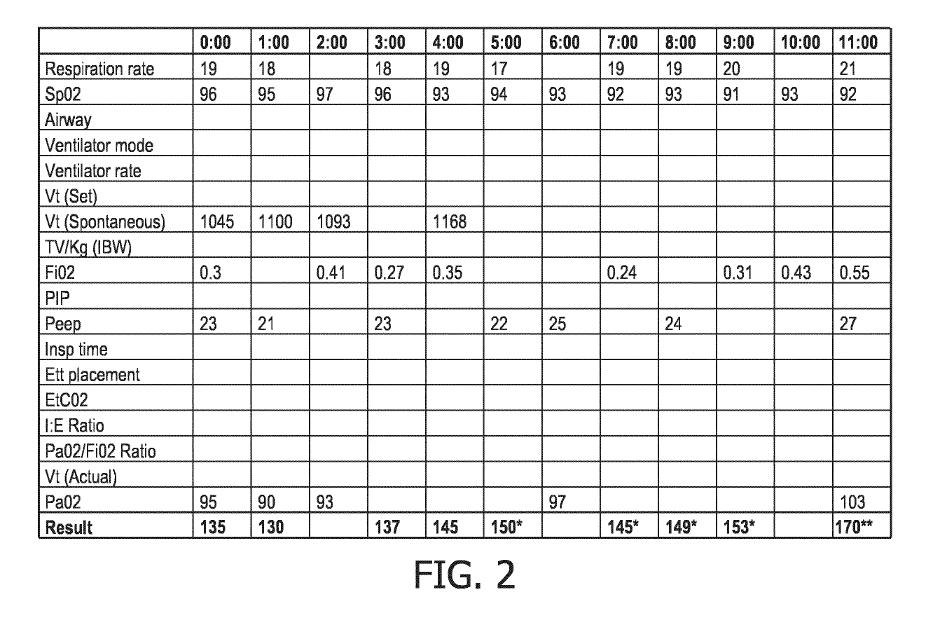

FIG. 2 depicts an example CDS interface that includes a table with most rows representing potential health parameter inputs and columns representing observation times. The bottommost row labeled “CDS Result” includes the results of the CDS algorithm that underlies FIG. 2. Assume calculation of the CDS Result is triggered at a particular point in time by receipt of an input for the patient's respiration rate. In this example, the patient's respiration rates were obtained each hour between 12:00 AM and 11:00 AM except for at 2:00 AM, 6:00 AM and 10:00 AM. Accordingly, CDS Results were calculated at each hour other than at 2:00 AM, 6:00 AM and 10:00 AM.

In this example (and other examples described herein), the CDS Results are dependent on five input health parameters in addition to the patient's Respiration Rate: blood oxygen saturation level (“SpO2”); tidal volume (“Vt”); fraction of inspired oxygen (“FiO2”); positive end-expiratory pressure (“PEEP”), and partial pressure of oxygen in blood (“PaO2”). However, this is not meant to be limiting, and it should be understood that other CDS algorithms may receive other health parameters as inputs in order to provide other CDS metrics. For this example, assume an input value is considered “fresh” if less than two hours old, “stale” if greater than two hours old but less than or equal to six hours old, and “expired” if greater than six hours old. Various additional criteria for determining such freshness will be apparent such as different thresholds, selection of thresholds based on the input value type (e.g., respiration rate input may become stale after 10 minutes while tidal volume may become stale after 2 hours), selection of thresholds based on the input value itself (e.g., a respiration rate of “30” may become stale after 2 minutes while a respiration rate of “20” may become stale after 10 minutes), or selection of thresholds based on other input values (SpO2 may become stale at different times depending on the current respiration rate a change observed therein). In some embodiments, appropriate time thresholds for judging an input value stale or expired may be statically defined, determined according to rules (e.g., interpreted if-then statements), or determined according to one or more machine learning algorithms (e.g., logistic regression models or neural networks) previously or continuously trained based on patient data.

In the “5:00” column, the CDS Result, “150,” is visually annotated with an asterisk (“*”). The asterisk indicates that the result is based on at least one value that is classified as “stale.” In particular, the most recently obtained PaO2 value was “93,” and was obtained three hours earlier at 2:00 AM. This asterisk therefore notifies a user that the CDS Result at 5:00 AM perhaps should be viewed with more scrutiny than other CDS values at other times because it was based at least in part on stale data. The asterisk may also indicate that, by obtaining a new PaO2, the CDS output may become more reliable. By contrast, in the “1:00” column, the CDS Result, “130,” is not visually annotated in spite of the fact that no FiO2 reading was available at 1:00 AM. That may be because the most recent FiO2 reading (“0.3”) was obtained just one hour earlier, which means that input is still considered “fresh,” and therefore the CDS Result in column 11:00 AM may not be deemed worthy of heightened scrutiny.

In the 7:00-9:00 AM columns, all three CDS Results (“145,” “149,” “153”) are annotated with asterisks. This may be because the patient's Vt (Spontaneous) was last obtained at 4:00 AM, which means these CDS Results are computed based at least in part on a stale input health parameter. At 11:00 am, there still has been no updated Vt (Spontaneous) reading taken from the patient. Because the 4:00 AM Vt (Spontaneous) reading is now expired (>6 hours old), a population average Vt (Spontaneous) may be used instead as input. Additional methods for inputting values for “missing” input values will be apparent may alternatively be employed in various embodiments. Consequently, the CDS result of “170” is visually annotated with two asterisks in signify to a user that this result should be subjected to a high level of scrutiny, since it is based at least in part on a population average, rather than on the patient's own health parameters. While not depicted in FIG. 2, in some embodiments, if a replacement input value is used as input for the CDS application, a corresponding field (e.g., the Vt (Spontaneous) cell in column 11:00 AM) may be populated with the replacement value, and in some instances, visually annotated in a same or different manner as the CDS Result calculated using that replacement value.

While in FIG. 2 and other figures herein, asterisks are used as examples of how various data points may be visually annotated to be presented more conspicuously than other data points, this is not meant to be limiting. Other types of visual or even audible annotations may be used to present data points more conspicuously than other data points. In some embodiments, in addition to or instead of annotating data points with asterisks, other characters, including numbers, letters, symbols, etc. may be used. Additionally or alternatively, in some embodiments, text may be annotated using various fonts, such as bold, italics, underlining, etc. Additionally or alternatively, in some embodiments, text may be annotated by coloring a background of the text, such as using various fill colors in the cells of FIG. 2. For example, stale input data points and/or CDS Results calculated based on stale input data points may be colored with one color, such as yellow. Expired and/or unavailable input data points, and/or CDS Results calculated based on replacement input data points (e.g., population averages, ranges, etc.) may be colored with another color, such as red. Additionally or alternatively, animations such as blinking text may be employed to draw attention to various data points. In some embodiments in which output of a CDS application are audibly rendered, any CDS Results (or inputs) may be called out as being “stale,” “expired,” “unavailable,” “fresh,” etc., to inform a doctor who is listening to the output of the reliability of the results. Further, it will be apparent that various embodiments may utilize interfaces other than that depicted in FIG. 2. For example, some alternative interfaces may not display input values and CDS outputs across a time period and, instead, may only output the “current” values. As another alternative, the current values (e.g, the right-most value in each row) may be displayed numerically, while a timeline of these values may be displayed as a running line chart. Such line charts and numerical values may be annotated for stale or out-of-date data. For example, a line color or background color of a line chart may gradually or abruptly change to a different color as the underlying value becomes stale or expired (and replaced with inputted data). In views these examples of added characters, added text, alternative color, alternative font characteristics, animation, etc., various similar approaches for presenting data points more conspicuously than other data points will be apparent.

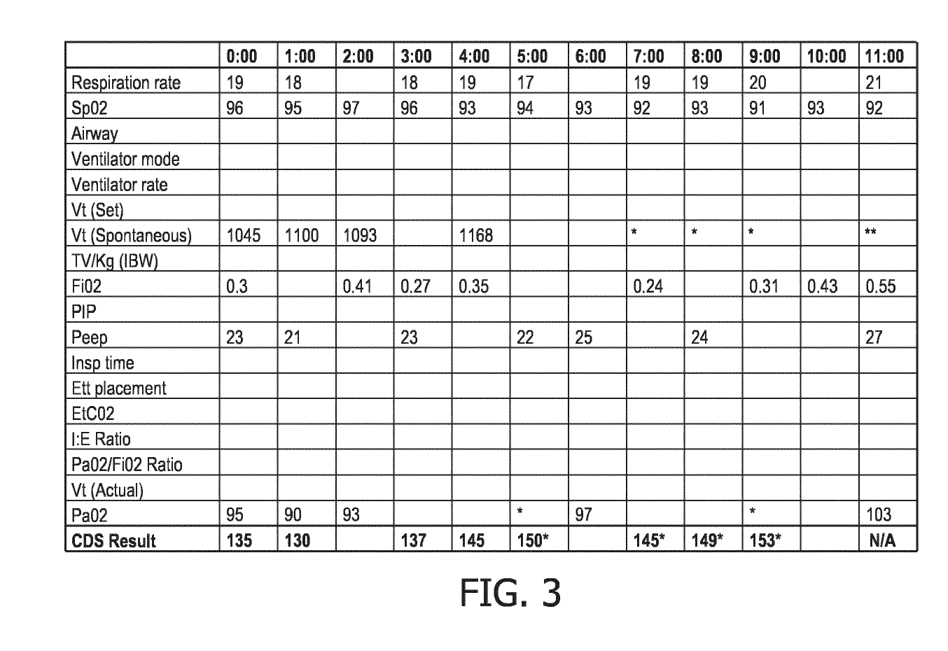

FIG. 3 depicts a CDS interface very similar to that depicted in FIG. 2. Once again, CDS Results that are calculated based on one or stale input health parameters are annotated with a single asterisk. However, in this example, input values that are stale are now also annotated with a single asterisk. For example, Vt (Spontaneous) is annotated with asterisks at columns 7-9:00 AM due to the last reading being obtained at 4:00 AM. Similarly, PaO2 is annotated with asterisks at columns 5:00 AM and 9:00 AM because the most recent PaO2 readings available at those times were at 2:00 AM and 6:00 AM, respectively. Additionally in FIG. 3, CDS Results are no longer available if any input health parameter is expired or otherwise unavailable. This is the case, for instance, with the CDS Result in the 11:00 AM column of FIG. 3, which is indicated to be “N/A” because the input value Vt (Spontaneous) is expired at that point, as indicated by the double asterisk annotation.

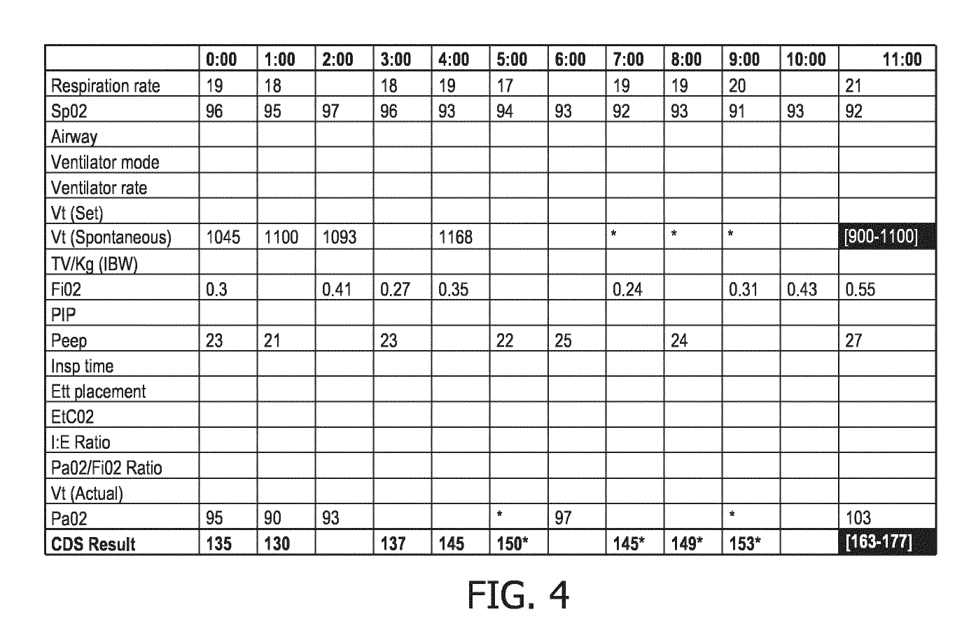

As noted above, in some embodiments, when a given input health parameter is unavailable or expired (e.g., more than six hours have elapsed since it was last measured), instead of replacing the input health parameter with a value such as a population average, other values may be used as replacements. For example, in some embodiments, a range of potential values may be used in place of the input value. An example of this is shown in FIG. 4, which depicts an interface similar to those depicted in FIGS. 2 and 3 except that a range of values, “900-1100,” is used for the missing Vt (Spontaneous) value in column 11:00 AM. To put a user on notice that a replacement range has been used (and hence, any CDS Results should be subject to heightened scrutiny), this range is visually annotated with the shading and the flanking brackets, “[ . . . ]” (though other annotations may be used instead). The corresponding CDS Result, “163-177,” is similarly annotated. Various ranges may be used as replacement values for an expired or unavailable health parameter. In some embodiments, for example, a replacement range may be one standard deviation around a mean population value.

The number of additional computations performed when using ranges in place of health parameter inputs may vary depending on a variety of factors, such as a number of calculations performed for the range, a number of unavailable/expired health parameter inputs, and so forth. Suppose an input health parameter is replaced with a range that starts one standard deviation below a mean population value for that parameter and stops at one standard deviation above the mean population value. Suppose further that two calculations are done, one for the mean-minus-standard deviation and one for the mean-plus-standard-deviation. If one health parameter input is unavailable or expired, this may result in two calculations, instead of the single calculation that may have been performed had the health parameter input been available. However, if two health parameter inputs are unavailable or expired, that may result in four calculations to cover all permutations. For example, the following equation may be employed when two input health parameters are unavailable or expired:

outₘᵢₙ=minimum(CDS(ν1+SD₁,ν2+SD₂),CDS(ν1+SD₁,ν2−SD₂),CDS(ν1−SD₁,ν2 +SD₂),CDS(ν1−SD₁,ν2−SD₂)

outₘₐₓ=maximum(CDS(ν1+SD₁,ν2+SD₂),CDS(ν1+SD₁,ν2−SD₂),CDS(ν1−SD₁,ν2 +SD₂),CDS(ν1−SD₁,ν2−SD₂)

wherein ν1 is the population mean for the first unavailable/expired input health parameter, SD₁, is the standard deviation for the first unavailable/expired input health parameter, ν2 is the population mean for the second unavailable/expired input health parameter, and SD₂ is the standard deviation for the second unavailable/expired input health parameter. In general, similar expanded equations may be used when more health parameters are unavailable or expired, so long as all permutations are checked.

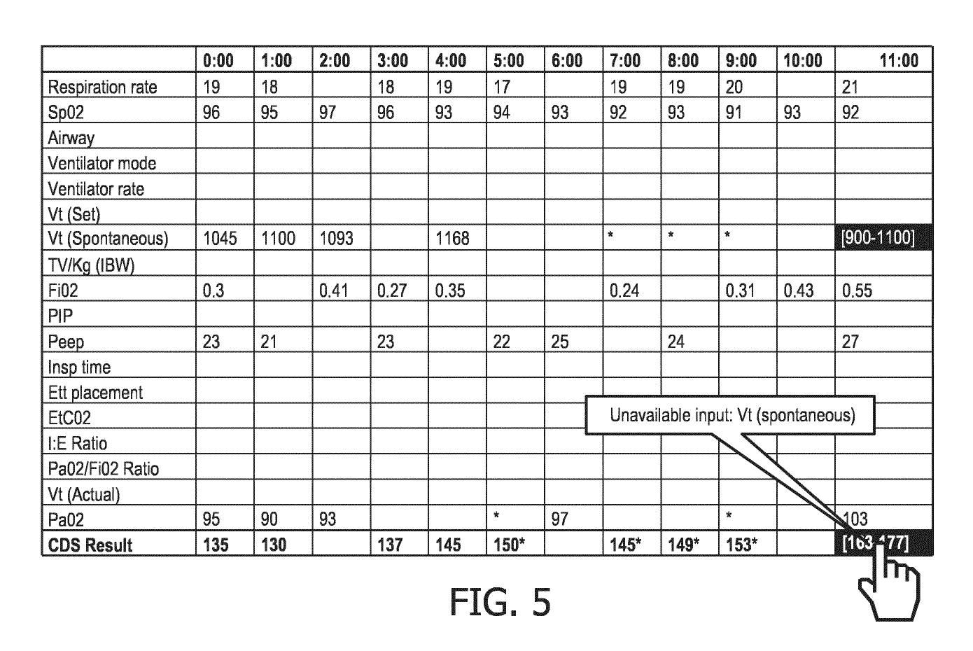

FIG. 5 depicts a CDS interface very similar to that depicted in FIG. 4. The main difference is that when the user hovers a cursor over the CDS result in the 11:00 AM column, a pop up window appears that notifies the user of the input health parameters for which a substitute value has been provided. In this example, the Vt (Spontaneous) input health parameter has been replaced with a range, 900-1100, which is annotated in the corresponding row of column 11:00 AM. However, in other embodiments, that additional annotation may be omitted or annotated differently. More generally, any of the visual annotations described herein may be used alone or in combination with any of the other visual annotations described herein. The examples in FIGS. 2-5 are not meant to be limiting, and are for illustrative purposes only.

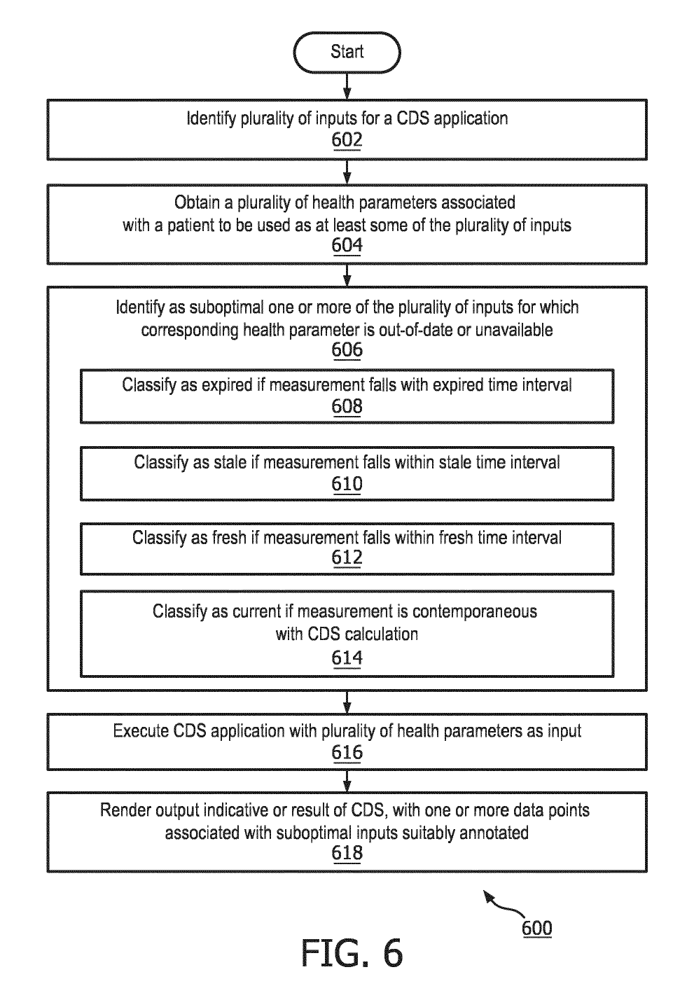

FIG. 6 depicts an example method 600 for visually and/or audibly annotating one or more input or output data points associated with execution of a CDS software application, in accordance with various embodiments. Although particular operations are shown in a specific order, this is not meant to be limiting. Various operations may be added, omitted, or reordered.

At block 602, a plurality of inputs for a CDS software application (or a CDS subroutine in a CDS application that provides a suite of CDS subroutines, each providing one or more different CDS metrics) may be identified, e.g., by CDS engine 102. For example, in the CDS algorithm repeatedly described above, the identified inputs would be SpO2, Vt (Spontaneous), FiO2, PEEP, and PaO2. At block 604, a plurality of health parameters associated with a patient for which a clinical decision is to be made may be obtained, e.g., by CDS engine 102. For example, CDS engine 102 may acquire various health parameters from health parameter engine 106 and/or from one or more medical apparatus 108. In some embodiments, and where applicable, when a medical apparatus 108 receives a request for a medical parameter from CDS engine 102, the medical apparatus 108 may respond by measuring the health parameter on the fly and returning the measured value to CDS engine 102, ensuring that the measured health parameter is “current.”

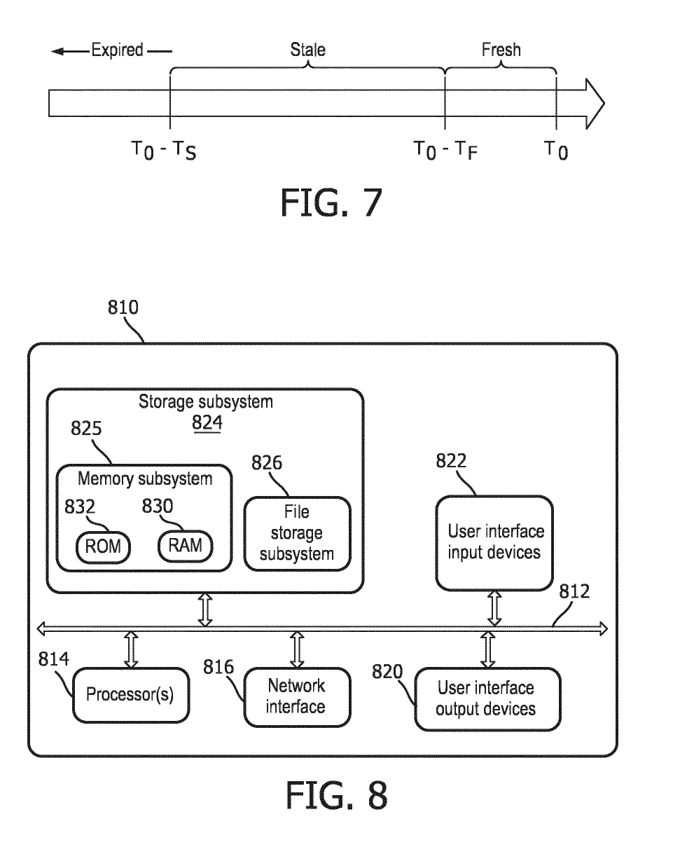

At block 606, one or more of the plurality of CDS application inputs identified at block 602 may be identified as suboptimal, e.g., by CDS engine 102, in response to corresponding health parameters of the patient being classified as expired/stale/unavailable, and in particular may be classified based on their position along a timeline such as that depicted in FIG. 7. In the timeline depicted in FIG. 7, T₀ is the present moment. T_{F} is the present (T₀) minus some relatively short time interval, such as two hours, during which health parameter measurements may be considered “fresh” because they are “good enough.” This time interval is labeled “FRESH” in FIG. 7. T_{S} is the present (T₀) minus a greater time interval than T_{F}, such as six hours. Health parameters measured between T₀-T_{F} and T₀-T_{S} may be considered out-of-date but “stale.” This time interval is labeled “STALE” in FIG. 7, and precedes the FRESH time interval. Stale health measurements may be used as inputs in a CDS algorithm, and CDS metrics computed based on stale health parameters may, in some cases, be more reliable that CDS metrics computed based on substitute values (e.g., population means, ranges). However, they may be less reliable than CDS metrics computed based on current or fresh health parameters. The time interval prior to the STALE time interval is labeled “EXPIRED” because any health parameters measured during this period are invalid because, for instance, their probative value is outweighed by their detrimental effect on the reliability of resulting CDS metrics. As explained above, expired health parameters may be replaced with substitute values.

Referring back to FIG. 6, at block 608, one or more health parameters of a patient may be classified as “expired” if the one or more health parameters were last measured during the EXPIRED time interval of FIG. 7. At block 610, one or more health parameters of a patient may be classified as “stale” if the one or more health parameters were last measured during the STALE time interval of FIG. 7. At block 612, one or more health parameters of a patient may be classified as “fresh” if the one or more health parameters were last measured during the FRESH time interval of FIG. 7. And at block 614, one or more health parameters of a patient may be classified as “current” if the one or more health parameters were measured concurrently and/or contemporaneously with execution of a CDS routine (see block 616).

At block 616, the CDS software application may be executed with the plurality of eligible health parameters obtained at block 604 (i.e., those parameters that are not expired or unavailable). For health parameters that are expired or otherwise unavailable (e.g., were not measured), substitute values such as population means or ranges may be used instead, as described above. At block 618, one or more results or metrics of the CDS application may be presented in visual and/or audible output, e.g., rendered by CDS engine 102 or one or more computing devices 110 operated by medical personnel. In various embodiments, the output may present one or more data points associated with CDS inputs identified as suboptimal at block 606 in a manner such that the one or more data points are suitably annotated to notify the user that those data points should be subject to heighted scrutiny. For example, one or more data points may be visually annotated with extra characters (e.g., asterisks), highlighting, font adjustment (e.g., bold, italic, underlined, increased or decreased font size, font face, etc.), animation, cell color and/or pattern fill, and so forth.

FIG. 8 is a block diagram of an example computer system 810. Computer system 810 typically includes at least one processor 814 which communicates with a number of peripheral devices via bus subsystem 812. As used herein, the term “processor” will be understood to encompass various devices capable of performing the various functionalities attributed to the CDS system described herein such as, for example, microprocessors, field-programmable gate arrays (“FPGAs”), application specific integrated circuits (“ASICs”), other similar devices, and combinations thereof. These peripheral devices may include a storage subsystem 824, including, for example, a memory subsystem 825 and a file storage subsystem 826, user interface output devices 820, user interface input devices 822, and a network interface subsystem 816. The input and output devices allow user interaction with computer system 810. Network interface subsystem 816 provides an interface to outside networks and is coupled to corresponding interface devices in other computer systems.

User interface input devices 822 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computer system 810 or onto a communication network.

User interface output devices 820 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computer system 810 to the user or to another machine or computer system.

Storage subsystem 824 stores programming and data constructs that provide the functionality of some or all of the modules described herein. For example, the storage subsystem 824 may include the logic to perform selected aspects of method 700, and/or to implement one or more aspects of CDS engine 102 or CDS clients operating on computing devices 110.

These software modules are generally executed by processor 814 alone or in combination with other processors. Memory 825 used in the storage subsystem can include a number of memories, including but not limited to a main random access memory (RAM) 830 for storage of instructions and data during program execution, a read only memory (ROM) 832 in which fixed instructions are stored, and other types of memories such as instruction/data caches (which may additionally or alternatively be integral with at least one processor 814). A file storage subsystem 826 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 826 in the storage subsystem 824, or in other machines accessible by the processor(s) 814. As used herein, the term “non-transitory computer-readable medium” will be understood to encompass both volatile memory (e.g. DRAM and SRAM) and non-volatile memory (e.g. flash memory, magnetic storage, and optical storage) but to exclude transitory signals.

Bus subsystem 812 provides a mechanism for letting the various components and subsystems of computer system 810 communicate with each other as intended. Although bus subsystem 812 is shown schematically as a single bus, alternative implementations of the bus subsystem may use multiple busses.

Computer system 810 can be of varying types including a workstation, server, computing cluster, blade server, server farm, or any other data processing system or computing device. In some embodiments, the computer system may be implemented within a cloud computing environment. For example, the CDS engine 102 may be implemented on one or more virtual machines running on hardware 810 within (or distributed among) one or more data centers. Due to the ever-changing nature of computers and networks, the description of computer system 810 depicted in FIG. 8 is intended only as a specific example for purposes of illustrating some implementations. Many other configurations of computer system 810 are possible having more or fewer components than the computer system depicted in FIG. 8.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope

1. A computer-implemented method, comprising:
identifying, by one or more processors, a plurality of inputs for a clinical decision support (“CDS”) algorithm that is executable by the one or more processors;
obtaining, by the one or more processors, a plurality of health parameters associated with a patient to be used as at least some of the plurality of inputs for the CDS algorithm;
identifying as suboptimal, by the one or more processors, one or more of the plurality of inputs for which a corresponding health parameter associated with the patient is out-of-date or unavailable;
executing, by the one or more processors, the CDS algorithm using the plurality of health parameters as input; and
rendering, by the one or more processors, output indicative of a result of the CDS algorithm, wherein the output presents at least one data point associated with the one or more suboptimal inputs more conspicuously than one or more data points associated with other inputs.

2. The computer-implemented method of claim 1, wherein the identifying as suboptimal comprises classifying a given health parameter of the patient as out-of-date or unavailable based on a comparison of a timestamp associated with the given health parameter with one or more time intervals.

3. The computer-implemented method of claim 2, wherein the one or more time intervals include a stale time interval, and the given health parameter is classified as stale when the timestamp associated with the given health parameter falls within the stale time interval.

4. The computer-implemented method of claim 3, wherein a health parameter classified as stale is used as input for the CDS algorithm, and wherein one or more data points associated with the stale health parameter are presented in a manner that reveals the stale classification.

5. The computer-implemented method of claim 4, wherein the one or more time intervals include a fresh time interval that follows the stale time interval, and the given health parameter is classified as fresh when the timestamp associated with the given health parameter falls within the fresh time interval.

6. The computer-implemented method of claim 5, wherein a health parameter classified as fresh is used as input for the CDS algorithm, and wherein one or more data points associated with the fresh health parameter are presented in a manner that differs from the stale health parameter.

7. The computer-implemented method of claim 3, wherein the one or more time intervals include an expired time interval that precedes the stale time interval, and the given health parameter is classified as expired when the timestamp associated with the given health parameter falls within the expired time interval.

8. The computer-implemented method of claim 7, wherein a substitute value is used as input for the CDS algorithm in place of a health parameter classified as expired, and wherein one or more data points associated with the expired health parameter are presented in a manner that reveals the expired classification.

9. The computer-implemented method of claim 8, wherein the substitute value comprises a population mean or a range of values that encompasses the mean.

10. The computer-implemented method of claim 1, wherein the identifying as suboptimal comprises classifying a given health parameter of the patient as unavailable based on a determination that the given health parameter has not been measured, wherein a substitute value is used as input for the CDS algorithm in place of a health parameter classified as unavailable, and wherein one or more data points associated with the unavailable health parameter are presented in a manner that reveals the unavailable classification.

11. The computer-implemented method of claim 1, wherein the output visually annotates the at least one data point associated with the one or more suboptimal inputs.

12. The computer-implemented method of claim 11, wherein the output visually annotates the at least one data point with highlighting, font selection, animation, or extra characters.

13. The computer-implemented method of claim 1, wherein the at least one data point associated with the one or more suboptimal inputs includes a value computed by the CDS algorithm based at least in part on the one or more suboptimal inputs.

14. The computer-implemented method of claim 1, wherein the identifying as suboptimal comprises classifying a given health parameter of the patient as expired or unavailable, and wherein a substitute range of values is used as input for the CDS algorithm in place of the given health parameter.

15. A clinical decision support (“CDS”) system comprising:
one or more processors; and
memory operably coupled with the one or more processors, wherein the memory stores instructions that, in response to execution of the instructions by the one or more processors, cause the one or more processors to perform the following operations:
identifying a plurality of inputs for a CDS algorithm that is executable by the one or more processors;
obtaining a plurality of health parameters associated with a patient to be used as at least some of the plurality of inputs for the CDS algorithm;
identifying as suboptimal one or more of the plurality of inputs for which a corresponding health parameter associated with the patient is out-of-date or unavailable;
executing the CDS algorithm using the plurality of health parameters as input; and
causing output indicative of a result of the CDS algorithm to be rendered, wherein the output presents at least one data point associated with the one or more suboptimal inputs more conspicuously than one or more data points associated with other inputs.

16. The system of claim 15, wherein the identifying as suboptimal comprises classifying a given health parameter of the patient as out-of-date or unavailable based on a comparison of a timestamp associated with the given health parameter with one or more time intervals.

17. The system of claim 16, wherein the one or more time intervals include a stale time interval, and the given health parameter is classified as stale when the timestamp associated with the given health parameter falls within the stale time interval.

18. The system of claim 17, wherein a health parameter classified as stale is used as input for the CDS algorithm, and wherein one or more data points associated with the stale health parameter are presented in a manner that reveals the stale classification.

19. The system of claim 18, wherein the one or more time intervals include a fresh time interval that follows the stale time interval, and the given health parameter is classified as fresh when the timestamp associated with the given health parameter falls within the fresh time interval.

20. The system of claim 19, wherein a health parameter classified as fresh is used as input for the CDS algorithm, and wherein one or more data points associated with the fresh health parameter are presented in a manner that differs from the stale health parameter.

21. The system of claim 17, wherein the one or more time intervals include an expired time interval that precedes the stale time interval, and the given health parameter is classified as expired when the timestamp associated with the given health parameter falls within the expired time interval.

22. The system of claim 21, wherein a substitute value is used as input for the CDS algorithm in place of a health parameter classified as expired, and wherein one or more data points associated with the expired health parameter are presented in a manner that reveals the expired classification.

23. The system of claim 22, wherein the substitute value comprises a population mean or a range of values that encompasses the mean.

24. The system of claim 15, wherein the identifying as suboptimal comprises classifying a given health parameter of the patient as unavailable based on a determination that the given health parameter has not been measured, wherein a substitute value is used as input for the CDS algorithm in place of a health parameter classified as unavailable, and wherein one or more data points associated with the unavailable health parameter are presented in a manner that reveals the unavailable classification.

25. The system of claim 15, wherein the output visually annotates the at least one data point associated with the one or more suboptimal inputs.

26. The system of claim 25, wherein the output visually annotates the at least one data point with highlighting, font selection, animation, or extra characters.

27. The system of claim 15, wherein the at least one data point associated with the one or more suboptimal inputs includes a value computed by the CDS algorithm based at least in part on the one or more suboptimal inputs.

28. The system of claim 15, wherein the identifying as suboptimal comprises classifying a given health parameter of the patient as expired or unavailable, and wherein a substitute range of values is used as input for the CDS algorithm in place of the given health parameter.

29. At least one non-transitory computer-readable medium comprising instructions that, in response to execution of the instructions by one or more processors, cause the one or more processors to perform the following operations:
identifying a plurality of inputs for a clinical decision support (“CDS”) algorithm that is executable by the one or more processors;
obtaining a plurality of health parameters associated with a patient to be used as at least some of the plurality of inputs for the CDS algorithm;
identifying as suboptimal one or more of the plurality of inputs for which a corresponding health parameter associated with the patient is out-of-date or unavailable;
executing the CDS algorithm using the plurality of health parameters as input; and
rendering output indicative of a result of the CDS algorithm, wherein the output presents at least one data point associated with the one or more suboptimal inputs more conspicuously than one or more data points associated with other inputs.

30. The at least one non-transitory computer-readable medium of claim 29, wherein the identifying as suboptimal comprises classifying a given health parameter of the patient as out-of-date or unavailable based on a comparison of a timestamp associated with the given health parameter with one or more time intervals.

31. The at least one non-transitory computer-readable medium of claim 30, wherein the one or more time intervals include a stale time interval, and the given health parameter is classified as stale when the timestamp associated with the given health parameter falls within the stale time interval.

32. The at least one non-transitory computer-readable medium of claim 31, wherein a health parameter classified as stale is used as input for the CDS algorithm, and wherein one or more data points associated with the stale health parameter are presented in a manner that reveals the stale classification.

33. The at least one non-transitory computer-readable medium of claim 32, wherein the one or more time intervals include a fresh time interval that follows the stale time interval, and the given health parameter is classified as fresh when the timestamp associated with the given health parameter falls within the fresh time interval.

34. The at least one non-transitory computer-readable medium of claim 33, wherein a health parameter classified as fresh is used as input for the CDS algorithm, and wherein one or more data points associated with the fresh health parameter are presented in a manner that differs from the stale health parameter.

35. The at least one non-transitory computer-readable medium of claim 31, wherein the one or more time intervals include an expired time interval that precedes the stale time interval, and the given health parameter is classified as expired when the timestamp associated with the given health parameter falls within the expired time interval.

36. The at least one non-transitory computer-readable medium of claim 35, wherein a substitute value is used as input for the CDS algorithm in place of a health parameter classified as expired, and wherein one or more data points associated with the expired health parameter are presented in a manner that reveals the expired classification.

37. The at least one non-transitory computer-readable medium of claim 36, wherein the substitute value comprises a population mean or a range of values that encompasses the mean.

38. The at least one non-transitory computer-readable medium of claim 29, wherein the identifying as suboptimal comprises classifying a given health parameter of the patient as unavailable based on a determination that the given health parameter has not been measured, wherein a substitute value is used as input for the CDS algorithm in place of a health parameter classified as unavailable, and wherein one or more data points associated with the unavailable health parameter are presented in a manner that reveals the unavailable classification.

39. The at least one non-transitory computer-readable medium of claim 29, wherein the output visually annotates the at least one data point associated with the one or more suboptimal inputs.

40. The at least one non-transitory computer-readable medium of claim 39, wherein the output visually annotates the at least one data point with highlighting, font selection, animation, or extra characters.

41. The at least one non-transitory computer-readable medium of claim 29, wherein the at least one data point associated with the one or more suboptimal inputs includes a value computed by the CDS algorithm based at least in part on the one or more suboptimal inputs.

42. The at least one non-transitory computer-readable medium of claim 29, wherein the identifying as suboptimal comprises classifying a given health parameter of the patient as expired or unavailable, and wherein a substitute range of values is used as input for the CDS algorithm in place of the given health parameter.
